(19) United States
(12) Patent Application Publication
(10) Pub. No.: US 2010/0292579 A1
(43) Pub. Date: Nov. 18, 2010

(54) VEIN IMAGING APPARATUS, VEIN IMAGE INTERPOLATION METHOD, AND PROGRAM

(76) Inventor: Hideo SATO, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/774,034

(22) Filed: May 5, 2010

(30) Foreign Application Priority Data
May 14, 2009 (JP) P2009-117986

(51) Int. Cl. A61B 6/00

(52) U.S. Cl. 600/474

(57) ABSTRACT

An imaging element of a vein imaging apparatus according to the invention includes a vein image data generation region for generating image data of a vein based on a near-infrared light that was condensed by a lens array and that was scattered in the living body and transmitted through the vein, and a thermal noise output data generation region that includes a pixel shielded from light and generates a thermal noise output that is an output value output from the pixel shielded from light. The vein imaging apparatus according to the present invention measures thermal noise based on the thermal noise output data, and estimates an imaging temperature based on a measurement result of this thermal noise. The vein imaging apparatus according to the present invention performs interpolation processing of the image based on the estimated temperature during imaging process.

### BACKGROUND OF THE INVENTION

1. Field of the Invention

The present invention relates to a vein imaging apparatus, a vein image interpolation method, and a program.

2. Description of the Related Art

Biometric individual authentication is highly important technology for protecting the rights in the future network society. In commercial transactions conducted on the Internet in which money, contents, and rights can be stolen any time over the network by means of spoofing, biometric individual authentications particularly attract attention as a technique for protecting a field that may not be protected by encryption alone. However, a biometric individual authentication using fingerprint and iris may not solve the issue of counterfeiting. With regard to this issue, an individual authentication technique using a part of the vein pattern that may not be easily imaged from the outside is expected to be a biometric individual authentication for the next generation because of accuracy of determination and difficulty for counterfeiting and spoofing.

On the other hand, in developing an imaging method for taking a vein image, it was difficult to make an imaging device with a planar structure because the position of a light source is strictly restricted. In order to solve this issue, there has been suggested a method using a wide-angle lens and the like. However, even with this method, it is difficult to limit the distance between a finger and the imaging device, and the user is required to surely place the finger at the same distance. Therefore, the reproducibility of the authentication may not be ensured. A contact or non-contact device with a large sensor is ideal in principle, but the large sensor size results in increasing the cost due to expensive optical materials. Further, because thermal noise occurring in a sensor increases in proportional to the increase in the temperature, when the external temperature becomes high during imaging, there arises an issue in that the image quality of the image used for the authentication decreases and therefore the authentication accuracy decreases.

As described above, an imaging device is largely affected by the external temperature, and it is important to know the temperature of the imaging device. Japanese Patent Application Laid-Open No. 2006-349957 discloses a technique for providing an apparatus with temperature detection means for detecting a temperature of an optical member used in an exposure apparatus and temperature adjustment means for adjusting the temperature of the optical member so that the detected temperature stays within a predetermined range.

### SUMMARY OF THE INVENTION

However, when the apparatus is provided with the temperature detection means and the temperature adjustment means as disclosed in Japanese Patent Application Laid-Open No. 2006-349957, there arises an issue that it is difficult to produce the apparatus smaller.

In light of the foregoing, it is desirable to provide a vein imaging apparatus, a vein image interpolation method, and a program that make it possible to prevent deterioration of the image quality due to thermal noise and to produce the apparatus smaller.

According to an embodiment of the present invention, there is provided a vein imaging apparatus including a lens array including a plurality of light-receiving lenses disposed in an array, a near-infrared light emission source that is arranged at an end of the lens array and emits a near-infrared light to a part of a living body, an imaging element including a vein image data generation region for generating image data of a vein based on the near-infrared light that was condensed by the lens array and that was scattered in the living body and transmitted through the vein, and a thermal noise output data generation region that includes a pixel shielded from light and generates a thermal noise output that is an output value output from the pixel shielded from light, a thermal noise measuring unit that measures a magnitude of thermal noise based on the thermal noise output data output from the thermal noise output data generation region, a temperature estimation unit that estimates an imaging temperature at which imaging processing of the vein is performed based on the magnitude of the thermal noise measured by the thermal noise measuring unit, and a vein image interpolation unit that generates an image of the vein using the vein image data generated by the vein image data generation region, and performs interpolation processing of the image of the vein based on the imaging temperature estimated by the temperature estimation unit.

According to the above configuration, the thermal noise measuring unit measures a magnitude of thermal noise based on the thermal noise output data output by the thermal noise output data generation region. The temperature estimation unit estimates an imaging temperature at which the imaging processing of a vein is performed based on the magnitude of the thermal noise measured by the thermal noise measuring unit. The vein image interpolation unit generates an image of a vein using vein image data generated from the vein image data generation region, and performs interpolation processing of the image of the vein based on the imaging temperature estimated by the temperature estimation unit.

The vein image interpolation unit preferably performs at least one of integration processing of the image of the vein for a predetermined period and denoising processing of the image of the vein based on the imaging temperature estimated by the temperature estimation unit.

In the image element a plurality of pixels located in the vein image data generation region preferably correspond to one of the light-receiving lenses. The vein image interpolation unit preferably performs the interpolation processing using the vein image data output from pixels located around a pixel that output the vein image data used for generating the image of the vein.

The vein imaging apparatus may further include a thermal noise output preprocessing unit that performs preprocessing, for allowing the thermal noise measuring unit to quantitatively process the thermal noise, on the thermal noise output data output from the thermal noise output data generation region.

The thermal noise output preprocessing unit may perform at least one of accumulative processing for accumulatively adding the thermal noise output data for a predetermined period and peak processing on the thermal noise output data.

The vein imaging apparatus may further include a drive control unit that performs drive control of at least the image element. The drive control unit may control at least one of a light-receiving time and a frame rate of the image element based on the magnitude of the thermal noise measured by the thermal noise measuring unit.

The vein imaging apparatus may further include a vein pattern extraction unit that extracts a vein pattern from the image of the vein. The vein pattern extraction unit may change a filter characteristic of a filter used for extracting the vein pattern based on the imaging temperature estimated by the temperature estimation unit.

The vein imaging apparatus may further include a warning unit for giving a warning when the imaging temperature output by the temperature estimation unit is equal to or more than a predetermined threshold value.

According to another embodiment of the present invention, there is provided a vein image interpolation method including the steps of measuring a magnitude of thermal noise based on a thermal noise output data output from a thermal noise output data generation region of a vein imaging apparatus including a lens array including a plurality of light-receiving lenses disposed in an array, a near-infrared light emission source that is arranged at an end of the lens array and emits a near-infrared light to a part of a living body, and an image element including a vein image data generation region for generating image data of a vein based on the near-infrared light that was condensed by the lens array and that was scattered in the living body and transmitted through the vein and the thermal noise output data generation region that includes a pixel shielded from light and generates a thermal noise output that is an output value output from the pixel shielded from light, estimating an imaging temperature at which an imaging processing of the vein is performed based on the magnitude of the measured thermal noise, and generating an image of the vein using the vein image data generated by the vein image data generation region, and performing interpolation processing of the image of the vein based on the estimated imaging temperature.

According to another embodiment of the present invention, there is provided a program for causing a computer that controls a vein imaging apparatus to realize a thermal noise measuring function for measuring a magnitude of thermal noise based on a thermal noise output data output from a thermal noise output data generation region, a temperature estimation function for estimating an imaging temperature at which an imaging processing of a vein is performed based on the magnitude of the thermal noise measured by the thermal noise measuring function, and a vein image interpolation function for generating an image of the vein using vein image data generated by a vein image data generation region, and performing interpolation processing of the image of the vein based on the imaging temperature estimated by the temperature estimation function, where the vein apparatus includes a lens array including a plurality of light-receiving lenses disposed in an array, a near-infrared light emission source that is arranged at an end of the lens array and emits a near-infrared light to a part of a living body, and an image element including the vein image data generation region for generating image data of a vein based on the near-infrared light that was condensed by the lens array and that was scattered in the living body and transmitted through the vein and a thermal noise output data generation region that includes a pixel shielded from light and generates a thermal noise output that is an output value output from the pixel shielded from light.

As described above, according to the embodiments of the present invention, the apparatus can be produced smaller, and the deterioration of the image quality due to thermal noise can be prevented.

### BRIEF DESCRIPTION OF THE DRAWINGS

FIG. 1 is a block diagram for illustrating a configuration of a vein imaging apparatus according to a first embodiment of the present invention;

FIG. 2 is an explanatory diagram for illustrating a vein imaging apparatus according to the embodiment;

FIG. 3 is an explanatory diagram for illustrating a vein imaging apparatus according to the embodiment;

FIG. 4A is an explanatory diagram for illustrating an image taken by a microlens array;

FIG. 4B is an explanatory diagram for illustrating an image taken by a microlens array;

FIG. 5 is an explanatory diagram for illustrating an imaging element according to the embodiment;

FIG. 6A is an explanatory diagram for illustrating an imaging element according to the embodiment;

FIG. 6B is an explanatory diagram for illustrating an imaging element according to the embodiment;

FIG. 6C is an explanatory diagram for illustrating an imaging element according to the embodiment;

FIG. 7 is an explanatory diagram for illustrating an imaging element according to the embodiment;

FIG. 8 is an explanatory diagram for illustrating a pixel selection unit according to the embodiment;

FIG. 9 is an explanatory diagram for illustrating a method for obtaining data from a particular pixel;

FIG. 10 is an explanatory diagram for illustrating a method for obtaining data from a particular pixel;

FIG. 11 is a flow diagram for illustrating a positional displacement interpolation method according to the embodiment; and

FIG. 12 is a block diagram for illustrating a configuration of a vein imaging apparatus according to each embodiment of the present invention.

### DETAILED DESCRIPTION OF THE EMBODIMENT(S)

Hereinafter, preferred embodiments of the present invention will be described in detail with reference to the appended drawings. Note that, in this specification and the appended drawings, structural elements that have substantially the same function and structure are denoted with the same reference numerals, and repeated explanation of these structural elements is omitted.

The description will be given in the following order.

(1) Object

(2) First Embodiment

(1-1) Regarding Configuration of Vein Imaging Apparatus
Regarding configuration of imaging unit
Regarding example of structure of imaging unit
Regarding image obtained by microlens array
Regarding Imaging element
Regarding configuration of image processing unit
Regarding configuration of authentication processing unit
Regarding obtaining data from particular pixel

(1-2) Regarding Vein Image Interpolation Method

(2) Regarding hardware configuration of vein imaging apparatus according to each embodiment of the present invention

(3) Summary

### <Object>

Before describing the vein imaging apparatus according to each embodiment of the present invention and the vein image interpolation method, the object of the present invention will be first described with an explanation the overview of the vein imaging apparatus.

In biometric authentication, especially in vein authentication, a method using a camera using as imaging element a Charge Coupled Device (CCD), a Complementary Metal Oxide Semiconductor (CMOS), and the like has been mainly used. Such vein authentication apparatus, however, is larger than that for a finger authentication, and accordingly, is applied to a limited range of applications.

In view of this circumstance, in the below-described vein imaging apparatus according to each embodiment of the present invention, a microlens array (MLA), i.e., a type of lens array and large sensor are used, which allows the vein imaging apparatus to be produced thinner.

Further, the large-sized sensor device is made of silicon and the like, and in a device using silicon, thermal noise increases according to the increase in the temperature. Especially, in a sensor device formed with a thin film such as a TFT sensor a noise at high temperature occurs remarkably, which greatly deteriorates a taken vein image. In addition, due to an increasing deterioration of the vein image, the authentication accuracy at high temperature might decrease.

In view of the above issues, the below-described vein imaging apparatus according to each embodiment of the present invention aims to automate correction by means of image processing and optimum sampling of each member at each temperature, by using pixels surrounding an imaging region of the imaging element to measure thermal noise.

### First Embodiment

### Regarding Configuration of Vein Imaging Apparatus

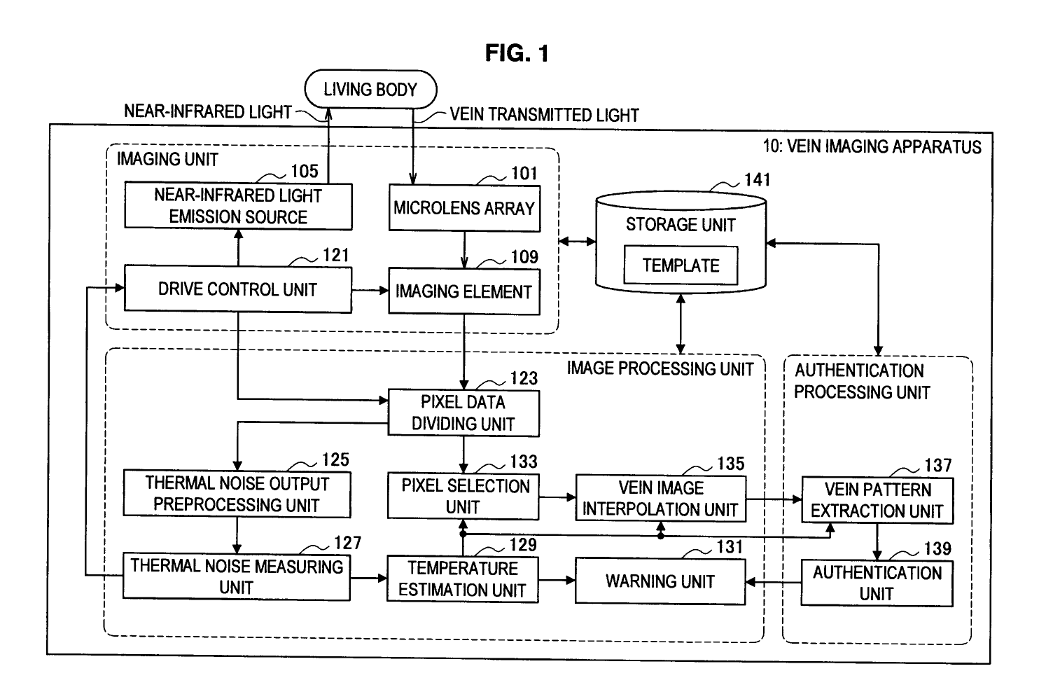

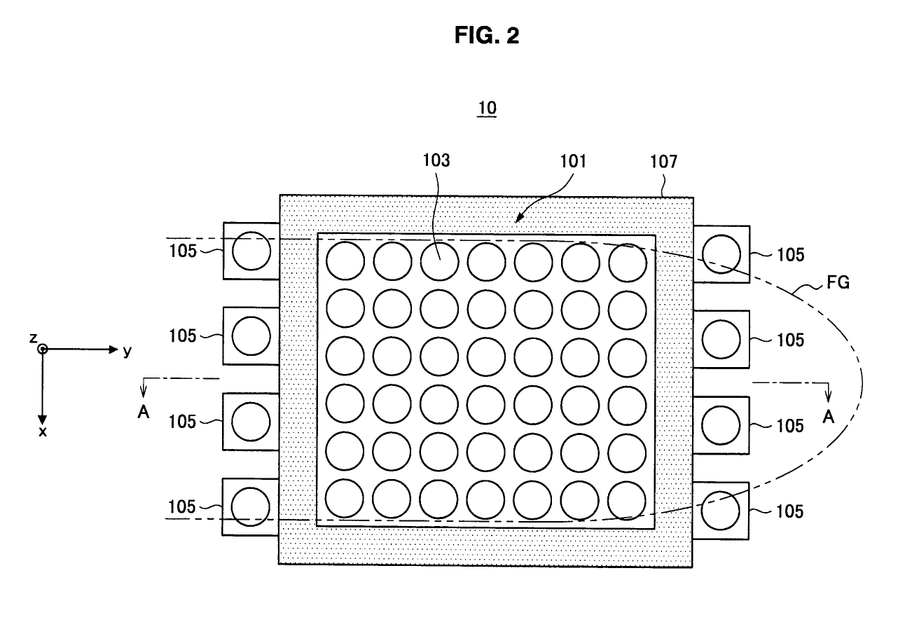

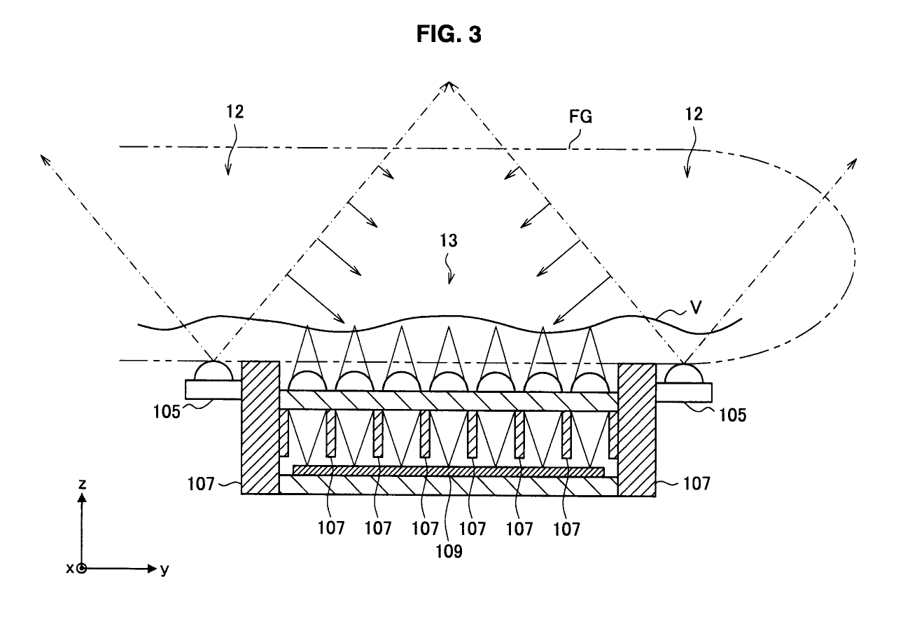

First, the configuration of the vein imaging apparatus according to the first embodiment of the present invention will be described in detail with reference to FIG. 1 to FIG. 3. FIG. 1 is a block diagram for illustrating the configuration of the vein imaging apparatus according to the present embodiment. FIG. 2 is a plan view of the vein imaging apparatus according to the present embodiment. FIG. 3 is a cross sectional diagram taken along line A-A of FIG. 2.

As shown in FIG. 1, the vein imaging apparatus 10 according to the present embodiment includes, for example, three units, i.e., an imaging unit, an image processing unit, and an authentication processing unit, and further includes a storage unit 141.

The imaging unit performs processing of imaging a part of a living body (for example, a finger). As shown in FIG. 1, this imaging unit mainly includes, for example, a microlens array 101, a near-infrared light emission source 105, an imaging element 109, and a drive control unit 121.

The image processing unit performs processing in acquisition of picture data (image data) related to a vein that is generated by the imaging unit, and various image processings on the obtained image data, and thus generates image (vein image) of a vein which is present inside of the living body. As shown in FIG. 1, this image processing unit mainly includes, for example, a pixel data dividing unit 123, a thermal noise output preprocessing unit 125, a thermal noise measuring unit 127, a temperature estimation unit 129, a warning unit 131, a pixel selection unit 133, a vein image interpolation unit 135.

The authentication processing unit performs authentication processing of the vein image generated by the image processing unit. As shown in FIG. 1, this authentication processing unit mainly includes, for example, a vein pattern extraction unit 137 and an authentication unit 139.

### [Regarding Configuration of Vein Imaging Apparatus]

First, the configuration of imaging unit will be hereinafter described in detail.

The microlens array (MLA) 101 condenses near-infrared light, which was emitted from the later-described near-infrared light emission source 105 to a part of a living body and transmitted through a vein inside the living body (which is also referred to hereinafter as vein transmitted light), onto the later-described imaging element 109. This microlens array 101 includes a plurality of light-receiving lens as described later. The microlens array 101 is made of, for example, a material that is more likely to be affected by heat than glass material. By using such material, it becomes possible to inexpensively bulk-produce microlens array of any size by means of, for example, molding. An example of such material that is more likely to be affected by heat than glass material includes a plastic resin.

The near-infrared light emission source 105 emits near-infrared light having a predetermined wavelength band onto a part of a living body placed on the vein imaging apparatus 10. Because the near-infrared light has characteristics that it is well transmitted through body tissues and absorbed by hemoglobin (reduced hemoglobin) in blood, if the near-infrared light is emitted on the finger, palm or back of a hand, veins distributed inside the finger, palm or back of the hand appear as a shadow in an image. The shadow of veins that appears in an image is called a vein pattern. In order to suitably image such a vein pattern, the near-infrared light emission source 105 emits near-infrared light having a wavelength of about 600 nm to 1300 nm or, preferably, about 700 nm to 900 nm.

If the wavelength of the near-infrared light emitted by the near-infrared light emission source 105 is less than 600 nm or more than 1300 nm, the percentage of light that is absorbed by hemoglobin in blood decreases, and it becomes difficult to obtain a suitable vein pattern. Also, if the wavelength of the near-infrared light emitted by the near-infrared light emission source 105 is about 700 nm to 900 nm, the near-infrared light is specifically absorbed by both deoxygenated hemoglobin and oxygenated hemoglobin, and it is therefore possible to obtain a suitable vein pattern.

As such a near-infrared light emission source 105, a light emitting diode (LED) may be used, for example. Further, in stead of using a light emitting diode having the above wavelength band, a combination of a light emitting diode capable of emitting light containing the above wavelength band and a filter for optically limiting the band of emitted light may be used. Further, the near-infrared light emission source 105 may be combined with an optical quantity adjustment filter that adjusts the distribution of light emitted by the light source.

For this near-infrared light emission source 105, emission timing of the near-infrared light and the intensity of the emitted near-infrared light and the like are controlled by the later-described drive control unit 121.

The imaging element 109 has an imaging surface with a plurality of pixels 111 arranged in a lattice structure, and generates vein image data with near-infrared light based on vein transmitted light focused by the microlens array 101. As the imaging element 109 according to the present embodiment can be used, for example, a CCD-image sensor, a CMOS-image sensor, a Thin Film Transistor (TFT)-image sensor, and the like. The imaging element 109 outputs the generated vein image data. Further, the imaging element 109 may record the generated vein image data in the later-described storage unit 141.

Besides, in the vein imaging apparatus 10 according to the present embodiment, the plurality of pixels 111 are assigned to one light-receiving lens of the microlens array 101 as described later. Therefore, in the vein imaging apparatus 10 according to the present embodiment, the near-infrared light (vein transmitted light) condensed by the one light-receiving lens is imaged with the plurality of pixels 111.

The Pixel scanning timing and the like of this imaging element 109 are controlled by the later-described drive control unit 121.

The drive control unit 121 can be realized by, for example, a CPU (Central Processing Unit), a ROM (Read Only Memory), a RAM (Random Access Memory), and the like. The drive control unit 121 performs drive control of the near-infrared illumination source 105 and the imaging element 109. Further, the drive control unit 121 adjusts the driving control of at least an imaging element 109 based on information about the magnitude of the thermal noise transmitted from the later-described thermal noise measuring unit 127. More specifically, the drive control unit 121 performs driving control of a light-receiving time (shutter speed) of the imaging element 109 and a frame rate of the imaging element 109 based on a predetermined synchronization signal. The drive control unit 121 can perform drive control related to the emission timing of the near-infrared light and the emission intensity of the near-infrared illumination source 105.

More specifically, regarding the control of the imaging element 109, the drive control unit 121 performs drive control along a certain direction of the imaging element 109 as such drive control of the imaging element 109 in which pixels along the certain direction are controlled in units of the number of pixels. In other words, on a cutaway view taken along a certain direction of the imaging element 109 according to the present embodiment, the imaging element 109 is considered to include, for example, seven pixels. In this case, the drive control unit 121 performs the driving control by dividing the pixels into seven groups in a direction along this cutting-plane line.

In controlling the near-infrared light emission source 105 and the imaging element 109, the drive control unit 121 can reference various parameters and databases recorded in the later-described storage unit 141.

### [Example of Structure of Imaging Unit]

Next, an example of a structure of the imaging unit according to the present embodiment will be described in detail with reference to FIG. 2 to FIG. 6C.

The microlens array 101 of the vein imaging apparatus 10 according to the present embodiment includes, for example, the plurality of microlenses 103, i.e., light-receiving lens as shown in FIG. 2, and the microlenses 103 are arranged in a lattice-pattern on a predetermined board. Each microlens 103 guides vein transmitted light that entered the microlens 103 through an incidence plane to the imaging element 109 (specifically, the pixels 111 of the imaging element 109), which is described later, as shown in FIG. 3, for example. The microlens array 101 is a lens array with a small curvature of field and with no distortion in the depth direction, and therefore suitable image data can be obtained by using such a microlens array 101. The focal position of each microlens 103 constituting the microlens array 101 is set at the position of a vein layer where a vein V exists, which is an imaging target of the vein imaging apparatus 10.

Human skin is known to have a three-layer structure including an epidermis layer, a dermis layer and a subcutaneous tissue layer, and the above-described vein layer exists in the dermis layer. The dermis layer is located at about 0.1 mm to 0.3 mm below the finger surface and has a thickness of about 2 mm to 3 mm. Thus, by setting the focal position of the microlens 103 at the position where the dermis layer exists (e.g. at the position that is about 1.5 mm to 2.0 mm below the finger surface), it is becomes possible to efficiently condense the light transmitted through the vein layer.

Besides, the number of the microlenses 103 disposed in the microlens array 101 according to the embodiment is not limited to the example shown in FIG. 2. The number of the microlenses 103 disposed in the microlens array 101 according to the embodiment may be set freely according to the size of a living body to be imaged, the size of the imaging element 109 or the like.

A plurality of light emitting diodes, which are an example of the near-infrared light emission source 105, are arranged at the opposed ends of the microlens array 101 as shown in FIG. 2, for example. The ends at which the light emitting diodes are arranged preferably correspond to the upper end and the lower end of a part of a living body (which is a finger FG in the example shown in FIGS. 2 and 3). By arranging the light emitting diodes in this manner, it is becomes possible to emit the near-infrared light from the upward and downward direction of the finger FG.

Besides, the number of the near-infrared light emission sources 105 according to the embodiment is not limited to the example shown in FIG. 2, and it may be set freely according to the size of the microlens array 101, an emission area of the near-infrared light emission sources 105 or the like.

Further, a directivity control plate 107 is placed between the microlens array 101 and the near-infrared light emission source 105 as shown in FIGS. 2 and 3, for example. This directivity control plate 107 controls the directivity of direct light 12 that is emitted from the near-infrared light emission sources 105 in such a way that the direct light 12 does not directly enter the microlenses 103 of the microlens array 101.

The near-infrared light that is emitted from the near-infrared light emission sources 105 propagates upward to the surface of the finger FG and enters the finger FG as the direct light 12 as shown in FIG. 3, for example. Because a human body is a suitable scatterer of near-infrared light, the direct light 12 that entered the finger FG propagates while scattering in all directions. A part of such scattered light travels as rear scattering light 13 through the above-described vein layer from the backside to the finger surface, and passes through the vein V on its way. The vein transmitted light that passed through the vein enters the respective microlenses 103 constituting the microlens array 101.

Here, the directivity control plate 107 is placed at the boundary between the adjacent microlenses 103. This directivity control plate 107 makes it possible to control of the directivity of the vein transmitted light, and the light that entered each microlens 103 can be separated from the adjacent microlenses 103. Accordingly, in the vein imaging apparatus 10 according to the embodiment, it becomes possible to select the vein transmitted light to be condensed on the imaging element 109 (specifically, the pixel 111).

### [Regarding Image Obtained by Microlens Array]

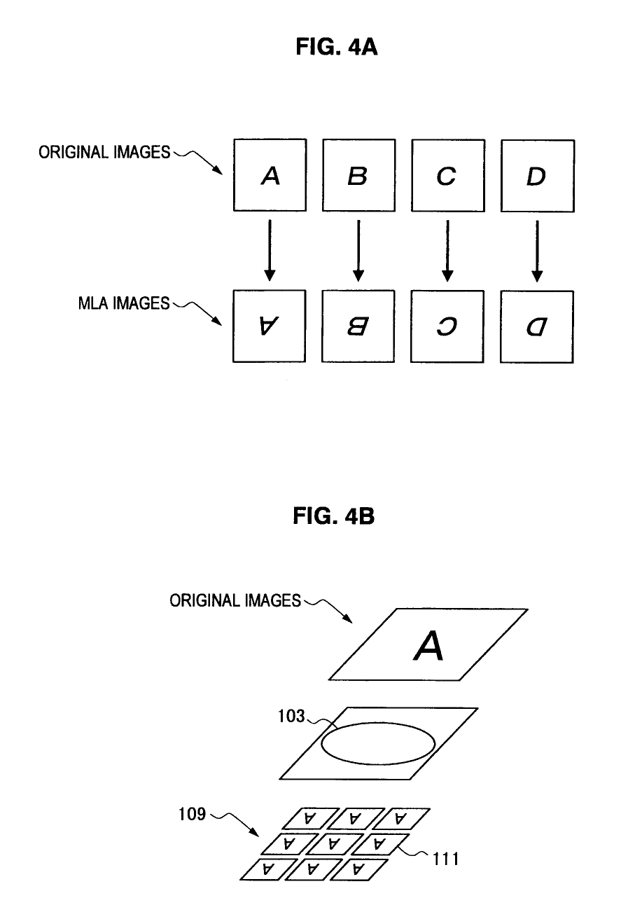

Next, the feature of the images obtained by the microlens array will be described in detail with reference to FIG. 4A and FIG. 4B. FIG. 4A and FIG. 4B are explanatory diagram for illustrating images taken by the microlens array.

Generally, if a certain image is taken by using a microlens array, a taken image is such a image whose up and down side and left and right side are respectively reversed from an original image as shown in FIG. 4A, for example. Further, because a plurality of pixels 111 are assigned to one light-receiving lens (microlens 103), an image whose up and down side and left and right side are reversed is created for all the pixels 111 that are assigned to one microlens 103. For example, if nine (3×3) pixels 111 are assigned to one microlens 103 as shown in FIG. 4B, an image whose up and down side and left and right side are reversed is created for each of the nine pixels 111.

As described later, the vein imaging apparatus 10 according to the present embodiment performs interpolation processing of images using image data generated by each of the plurality of pixels 111 corresponding to one of the microlenses 103.

### [Regarding Imaging Element]

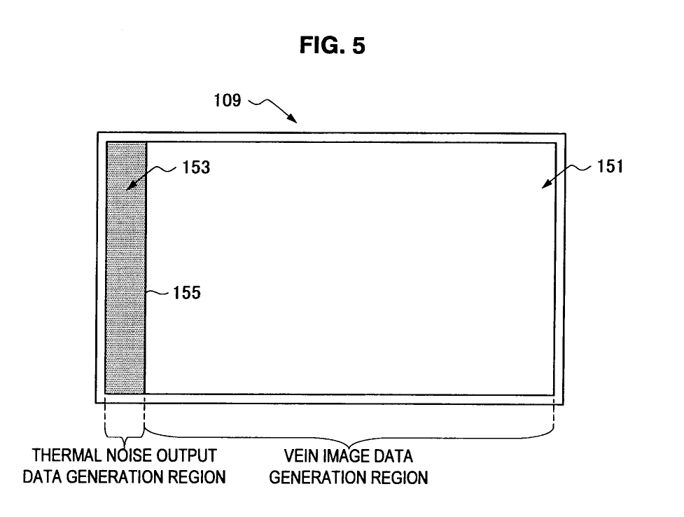

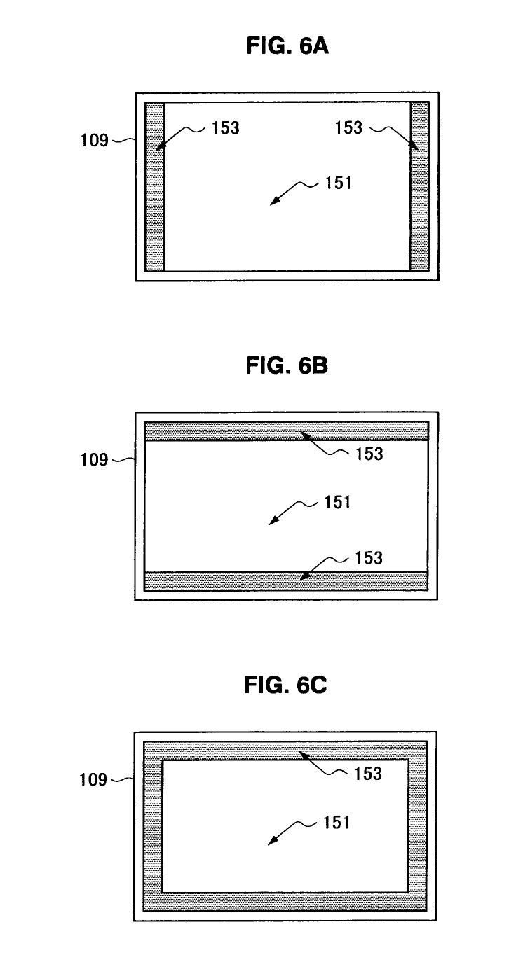

Next, the imaging element 109 of the vein imaging apparatus 10 according to the present embodiment will be described in detail with reference to FIG. 5, FIG. 6A to FIG. 6C. FIG. 5, FIG. 6A to FIG. 6C are explanatory diagrams for illustrating the imaging element according to the present embodiment.

In the imaging element 109 of the vein imaging apparatus 10 according to the present embodiment, a region in which the pixels 111 are formed in the imaging element 109 is divided into, for example, two regions 151 and 153 as shown in FIG. 5. The one region 151 is a vein image data generation region used to generate vein image data. The other region 153 is a thermal noise output data generation region for generating thermal noise output data that are used to estimate the magnitude of thermal noise.

In the vein image data generation region 151 a plurality of pixels (not shown) is arranged in an array, and the vein transmitted light condensed by the plurality of microlenses 103 of the microlens array 101 reaches the pixels 111. The vein image data output by the vein image data generation region 151 are data related to the intensity of the light detected by the pixels that generated the image data.

In the thermal noise output data generation region 153 a plurality of pixels (not shown) is arranged in an array. As shown in FIG. 5, the thermal noise output data generation region 153 is shielded by, for example, a shielding film 155, so that the external light does not enter the thermal noise output data generation region 153. Therefore, the data output from this thermal noise output data generation region 153 is not the data generated as the detection result of the external light by the pixels included in this region, but data related to thermal noise occurring depending on the temperature in the apparatus or the external temperature. The size of the thermal noise output data generation region 153 arranged on this imaging element 109 can be determined according to the size of the microlens array 101 used together with the imaging element 109.

For example, the above-described thermal noise output data generation region 153 may be arranged at one end of the imaging element 109 along one side of the imaging element 109 as shown in FIG. 5. Alternatively, the thermal noise output data generation region 153 may be arranged along opposing sides of the imaging element 109 as shown in FIG. 6A and FIG. 6B, or may be arranged along four sides of the imaging element 109 as shown in FIG. 6C.

### [Configuration of Image Processing Unit]

Next, FIG. 1 is referenced again. The configuration of the image processing unit of the vein imaging apparatus 10 according to the present embodiment will be described in detail.

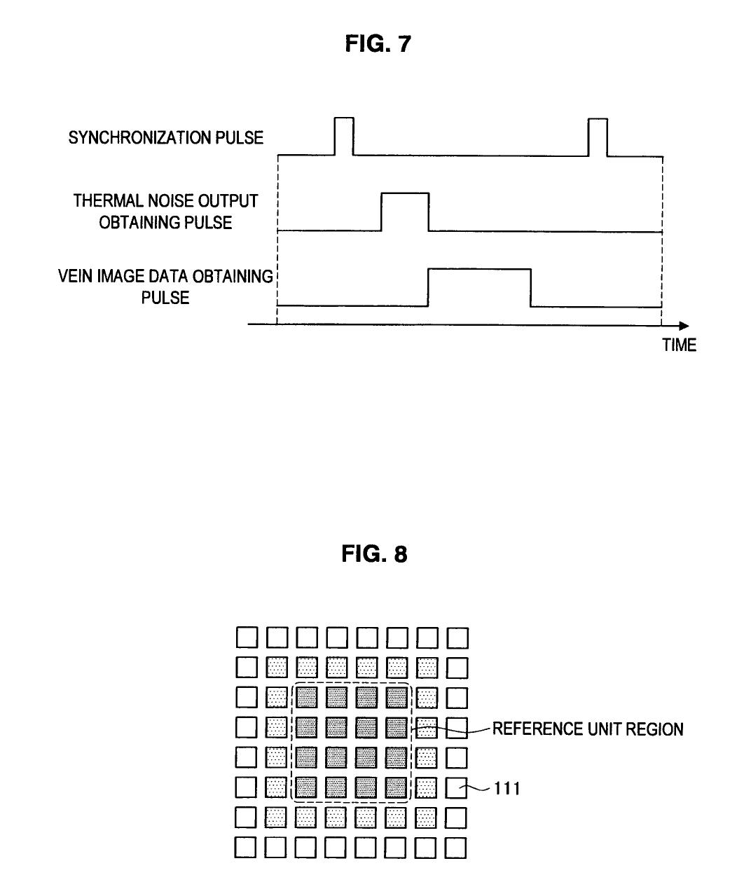

The pixel data dividing unit 123 is realized by, for example, CPU, ROM, and RAM. As shown in FIG. 7, for example, the pixel data dividing unit 123 determines, based on pulses for scanning the imaging element 109 input from the drive control unit 121, from which of the two regions of the imaging element 109 the pixel data transmitted from the imaging element 109 was output. As exemplified in FIG. 7, three kinds of pulses, namely, a pulse for synchronization in a vertical (or horizontal) direction of the imaging element, a thermal noise output obtaining pulse, a vein image data obtaining pulse are used in order to obtain outputs from the two regions of the imaging element 109. Therefore, the pixel data dividing unit 123 can determine according to these pulses, whether thermal noise output data are transmitted, or vein image data are transmitted.

The pixel data dividing unit 123 transmits to the later-described thermal noise output preprocessing unit 125 the data obtained during a period in which the thermal noise output obtaining pulse is in the Hi state (namely, thermal noise output data). Further, the pixel data dividing unit 123 transmits to the later-described pixel selection unit 133 the data obtained during a period in which the pulse for obtaining vein image data is Hi state (namely, vein image data).

The thermal noise output preprocessing unit 125 is realized by, for example, CPU, ROM, RAM, and the like. The thermal noise output preprocessing unit 125 performs preprocessing on thermal noise output data transmitted from the pixel data dividing unit 123 to allow the later-described thermal noise measuring unit 127 to quantitatively process the thermal noise. Examples of the above preprocessing include accumulation processing for accumulatively adding thermal noise output data for a predetermined period and a peak processing performed on the thermal noise output data. The thermal noise output preprocessing unit 125 carries out at least any one of the accumulation processing and the peak processing. Alternatively, the thermal noise output preprocessing unit 125 may carry out both of the accumulation processing and the peak processing, and may include preprocessing other than these processings.

The thermal noise output data output from thermal noise output data generation region 153 is an unstable output that varies over time. By performing the above processings, it is possible to stabilize numerical values representing thermal noise outputs included in the thermal noise output data to such extent that the numerical values can be quantitatively evaluated. Thereby, it becomes possible to improve the accuracy of the measuring processing performed by the later-described thermal noise measuring unit 127.

The thermal noise output generation unit 153 transmits the thermal noise output data on which the preprocessing was performed to the thermal noise measuring unit 127.

The thermal noise measuring unit 127 is realized by, for example, CPU, ROM, RAM, and the like. The thermal noise measuring unit 127 analyzes the thermal noise output data transmitted by the thermal noise output preprocessing unit 125, and measures the magnitude of the thermal noise occurring in the imaging element 109. The thermal noise measuring unit 127 transmits the measurement result of the thermal noise as thermal noise information to the drive control unit 121 and the temperature estimation unit 129.

The drive control unit 121 can reduce the driving frequency of the imaging element 109, and can control the light-receiving time (namely, shutter speed) and the frame rate of the imaging element 109 according to the magnitude of the thermal noise transmitted from the thermal noise measuring unit 127. As the thermal noise increases, an S/N (Signal to Noise) ratio of the data output from the imaging element 109 decreases. Accordingly, the drive control unit 121 can prevent the S/N ratio of the data signal caused by the thermal noise from decreasing, by reducing the driving frequency of the imaging element 109, increasing the light-receiving time, and slowing the frame rate.

The temperature estimation unit 129 is realized by, for example, CPU, ROM, RAM and the like. The temperature estimation unit 129 estimates the temperature (imaging temperature) at which the apparatus performs the imaging processing based on the thermal noise information, i.e., information about the magnitude of the thermal noise transmitted from the thermal noise measuring unit 127. Here, the temperature at which the vein imaging apparatus 10 performs the imaging processing may be an external temperature at the location in which the vein imaging apparatus 10 is installed, or may be a temperature that the vein imaging apparatus 10 attains. The temperature estimation unit 129 has a database containing correspondence relationship between the magnitude of the thermal noise occurring in the imaging element 109 and the temperature at which the imaging processing is performed. The temperature estimation unit 129 estimates based on this database the temperature from the magnitude of the thermal noise. The database may express this correspondence relationship by an expression representing the relationship between these two parameters.

For example, the above-described data base may be generated by measuring the magnitude of the occurring thermal noise while varying the temperature during the production of the vein imaging apparatus 10. The thus generated database includes the feature of the thermal noise specific to each of the vein imaging apparatuses 10, so that the relationship between the temperature and the thermal noise occurring in the apparatus can be accurately estimated.

The temperature estimation unit 129 can estimate the decease of the S/N ratio of the data signal output from the imaging element 109 based on the estimation result of the temperature. For example, the degree of this decrease of the S/N ratio can be estimated by generating a database, in advance, using results of evaluation experiments and the like for clarifying the relationship between the S/N ratio and the temperature at which the apparatus is assembled and by using the thus generated database.

The temperature estimation unit 129 transmits the estimation results of the temperature (for example, information indicating that the current temperature is 70 degrees Celsius) to the warning unit 131, the pixel selection unit 133, the vein image interpolation unit 135, and a vein pattern extraction unit 137, which are described later. When the temperature estimation unit 129 estimates, e.g., the degree the decrease of the S/N ratio, the temperature estimation unit 129 may transmit an estimation result (for example, information indicating that a decrease by about 10 dB is expected) as well as the estimation result of the temperature.

The warning unit 131 is realized by, for example, CPU, ROM, RAM and the like. The warning unit 131 references the estimation result of the temperature and the like transmitted from the temperature estimation unit 129, and when the temperature of the vein imaging apparatus 10 or the external temperature is equal to or more than a predetermined threshold value, the warning unit 131 determines that it is difficult to perform a normal vein imaging processing (furthermore the vein authentication processing), and accordingly outputs a warning.

Further, when the warning unit 131 receives, from the later-described authentication unit 139, information indicating that authentication of a vein pattern obtained from a certain user has failed for a predetermined number of times or more, the warning unit 131 may determine that the apparatus itself is under an environment that does not allow the apparatus to perform normal operation, and may output a warning accordingly.

Further, when the warning unit 131 determines that the temperature reaches such a level at which the apparatus may not perform a normal vein imaging processing (furthermore, the vein authentication processing), the warning unit 131 may stop the vein imaging processing and the vein authentication processing being carried out by the apparatus itself.

The pixel selection unit 133 is realized by, for example, CPU, ROM, RAM and the like. The pixel selection unit 133 selects a pixel for generating vein image data used to generate vein image from among the plurality of pixels 111 corresponding to one of the microlenses 103. The pixel selection processing performed by the pixel selection unit 133 will be hereinafter described with reference to FIG. 8. FIG. 8 is an explanatory diagram for illustrating the pixel selection processing performed by the pixel selection unit 133.

FIG. 8 shows a case where one microlenses 103 in the microlens array 101 corresponds to 8×8=64 pixels 111 and where the microlens 103 is a lens for reducing the size of the object by one half. In this case, the size of the object is reduced to one half the size. Accordingly, image data of the object can be obtained by using 4×4=16 pixels located in the central portion from among 64 pixels. Even in this case, the light from the object focuses on pixels other than pixels in the central portion, and the image data obtained from the portion other than the 4×4 pixels in the central portion can also be used to generate an object image.

At this occasion, a reference unit region serving as a reference unit of pixel selection is a region including 4×4=16 pixels as described above because of the magnification of the micro lens 103. Further, if there is no displacement of the image focus position and the like, the light condensed by the microlens 103 focuses on a substantially central portion of the 8×8=64 pixels. Accordingly, the pixel selection unit 133 selects 4×4 pixels located in a central portion from among 8×8 pixels corresponding to one of the microlenses 103.

Further, the pixel selection unit 133 references the estimation result of the temperature transmitted from the temperature estimation unit 129, and when the S/N ratio of the data signal output from the pixels 111 is expected to decrease, the pixel selection unit 133 also selects pixels that are located around the reference unit region and are detecting light.

The pixel selection unit 133 transmits, to the later-described vein image interpolation unit 135, the information about the thus selected pixels (for example, information for identifying the selected pixels) and the vein image data obtained from the selected pixels.

The vein image interpolation unit 135 is realized by, for example, CPU, ROM, RAM and the like. The vein image interpolation unit 135 generates the vein image based on the vein image data transmitted from the pixel selection unit 133. Further, the vein image interpolation unit 135 performs interpolation processing on the generated vein image based on the information about the temperature transmitted from the temperature estimation unit 129 (information representing the temperature, the estimation result about the decrease of the S/N ratio, and the like).

When generating the vein image, the vein image interpolation unit 135 corrects the output value included in the vein image data based on the information about the temperature transmitted from the temperature estimation unit 129. More specifically, the vein image interpolation unit 135 does not use the output value itself included in the vein image data to generate the image, but performs correction of the output value based on the following expression 1.

Corrected output value=(data output value−black level reference value)/(white level reference value−black level reference value) (Expression 1)

In the above expression 1, the black level reference value is an output value of data output from the imaging element 109 when the imaging element 109 images an image only in black color, and the white level reference value is an output value of data output from the imaging element 109 when the imaging element 109 images an image only in white color. In the above expression 1, the output value of data is an output value of data output by the imaging element 109 when the imaging element 109 images an object.

When the imaging temperature becomes high, the rate of the thermal noise increases with respect to the data output value, so that the contrast of the image decreases, and the entire image becomes whiter. In view of this issue, the vein image interpolation unit 135 according to the present embodiment changes based on the information about the temperature transmitted from the temperature estimation unit 129, the value of the black level reference value, and automatically prevents deterioration of the image caused by the increase of the thermal noise. More specifically, in the above expression 1, a correction coefficient is set by which the black level reference value at normal temperature is multiplied, and the vein image interpolation unit 135 adjusts the value of this correction coefficient based on the information about the temperature transmitted from the temperature estimation unit 129.

An example of the interpolation processing performed by the vein image interpolation unit 135 includes denoising processing of the generated vein image. Alternatively, the vein image interpolation unit 135 may perform integration processing on the plurality of frame images and perform processing for improving the image quality of the vein image, according to the information about the temperature transmitted from the temperature estimation unit 129. When the plurality of frame images are integrated, it takes more time to perform the processing, and the user of the vein imaging apparatus 10 has to wait for a longer time. However, it is possible to curb the occurrence of the situation where a vein may not be imaged due to an environmental temperature and the like (furthermore, the vein authentication processing may not be carried out).

Further, the vein image interpolation unit 135 may perform the following interpolation processing, for example, using a multi-tap interpolation filter according to the information about the temperature transmitted from the temperature estimation unit 129. As shown in FIG. 8, the vein image interpolation unit 135 may perform the interpolation processing (composition processing) of the vein image using not only the image data obtained from the pixels included in the reference unit region but also the image data obtained from the pixels that are located around the reference unit region and are detecting light. The above processing makes it possible to improve the image quality (S/N ratio) of the vein image even when the S/N ratio decreases as the thermal noise increases.

The vein image interpolation unit 135 transmits, to the later-described vein pattern extraction unit 135, the vein image on which the interpolation processing was performed.

### [Regarding Configuration of Image Processing Unit]

The vein pattern extraction unit 137 is realized by, for example, CPU, ROM, RAM and the like. The vein pattern extraction unit 137 has, for example, a function of performing preprocessing of the vein pattern extraction on the vein image transmitted from the vein image interpolation unit 135, a function of extracting the vein pattern, and a function of performing a postprocessing of the vein pattern extraction.

Examples of the above preprocessing of the vein pattern extraction include processing of detecting an outline of a finger from the vein image and recognizing at which position of the vein image the finger is located, and processing of rotating the taken image using the detected outline of the finger and correcting the angle of the taken image.

The above extraction of the vein pattern is performed by applying a differential filter to the taken image on which the outline detection processing and the angle correction processing are completed. The differential filter is a filter that outputs a larger value as an output value at a part where a difference between a pixel of interest and a neighboring pixel is large. In other words, the differential filter is a filter that enhances a line or an edge in an image by the operation using a difference in gradation value between a pixel of interest and pixels in its neighborhood.

Generally, if filtering is performed using a filter h(x, y) on image data u(x, y) with a lattice point (x, y) on a two-dimensional plane as a variable, image data v(x, y) is generated as represented by the following Expression 2. In Expression 2, “*” indicates convolution integral.

$$\begin{matrix}v ( x , y ) = u ( x , y ) * h ( x , y ) \\ = {\sum }_{{m}_{1}} {\sum }_{{m}_{2}} h ( {m}_{1} , {m}_{2} ) u ( x - {m}_{1} , y - {m}_{2} ) \\ = {\sum }_{{m}_{1}} {\sum }_{{m}_{2}} u ( {m}_{1} , {m}_{2} ) h ( x - {m}_{1} , y - {m}_{2} )\end{matrix} \qquad Expression 2$$

In the extraction of a vein pattern according to this embodiment, a differentiation filter such as a primary space differentiation filter or a secondary space differentiation filter may be used as the above-described differential filter. The primary space differentiation filter is a filter that calculates a difference in gradation value between a pixel of interest and an adjacent pixel in the horizontal direction and the vertical direction, and a secondary space differentiation filter is a filter that extracts, for a pixel of interest, a part where the amount of change in difference in gradation value is large.

As the secondary space differentiation filter, the following Laplacian of Gaussian (LOG) filter may be used. The LOG filter (Expression 4) is represented by a second order derivative of a Gaussian filter (Expression 3), which is a smoothing filter using the Gaussian function. In the following Expression 3, a indicates a standard deviation of the Gaussian function, which is a variable indicating the degree of smoothing of the Gaussian filter. Further, a in the following Expression 4 is a parameter indicating a standard deviation of the Gaussian function as in Expression 3, and an output value when performing LOG filtering can be changed by changing a value of σ.

$$\begin{matrix}{h}_{gauss} ( x , y ) = \frac{1}{2 {πσ}^{2}} exp \{ - \frac{( {x}^{2} + {y}^{2} )}{2 {σ}^{2}} \} & Expression 3 \\ \begin{matrix}{h}_{Log} ( x , y ) = {∇}^{2} \cdot {h}_{gauss} ( x , y ) \\ = ( \frac{{\partial }^{2}}{\partial {x}^{2}} + \frac{{\partial }^{2}}{\partial {y}^{2}} ) {h}_{gauss} \\ = \frac{( {x}^{2} + {y}^{2} - 2 {σ}^{2} )}{2 {πσ}^{6}} exp \{ - \frac{( {x}^{2} + {y}^{2} )}{2 {σ}^{2}} \}\end{matrix} & Expression 4\end{matrix}$$

Examples of the above postprocessing of the vein pattern extraction include threshold processing performed on the taken image to which the differential filter was applied, binarization processing, and thinning processing. After the above postprocessing, a skeleton of the vein pattern can be extracted.

The vein pattern extraction unit 137 according to the present embodiment may change the filter characteristics of the above-described filter (for example, coefficient values in each expression representing a filter) according to the information about the temperature transmitted from the temperature estimation unit 129. As described above, the vein image interpolation unit 135 performs appropriate image interpolation processing according to the increase of the thermal noise caused by high temperature. Due to this interpolation processing, there might occur the case where appropriate extraction processing of the vein pattern may not be performed with a filter that is still set in such a manner that it is used to extract the vein pattern at the normal temperature. In view of this issue, the vein pattern extraction unit 137 changes the filter characteristics according to the information about the temperature, and thereby it becomes possible to extract an appropriate vein pattern from even the image taken at high temperature.

The vein pattern extraction unit 137 transmits the vein pattern and the skeleton thus extracted to the later-described authentication unit 139. In addition, the vein pattern extraction unit 137 may store the extracted vein pattern and the skeleton in the later-described storage unit 141. The vein pattern extraction unit 137 may further store a parameter generated when performing each processing, the progress of processing or the like in the storage unit 141.

The authentication unit 139 is realized by, for example, CPU, ROM, RAM and the like. The authentication unit 139 authenticates the vein pattern by collating the vein pattern generated by the vein pattern extraction unit 137 with an already-registered template.

The vein pattern authentication unit 139 authenticates the generated vein pattern based on the vein pattern that is generated by the vein pattern extraction unit 137 and the template of the vein pattern that has been registered. The vein pattern authentication unit 139 requests the storage unit 141, which is described later, to disclose the registered vein pattern and compares the acquired registered vein pattern with the vein pattern transferred from the vein pattern extraction unit 137. The comparison between the registered vein pattern and the transferred vein pattern can be carried out based on a correlation coefficient, which is calculated as follows, for example. In a case where, as a result of the comparison, the registered vein pattern is determined to be similar to the transmitted vein pattern, the authentication unit 139 determines that the authentication of the transmitted vein pattern is successful. When the registered vein pattern is determined not to be similar to the transmitted vein pattern, the authentication unit 137 determines that the authentication has failed.

The correlation coefficient is defined by the following Expression 5, and it is a statistical indicator that indicates a similarity between two data x={xi} and y={yi}, which is a real value from −1 to 1. If the correlation coefficient indicates a value close to 1, it means that the two data are similar, and if the correlation coefficient indicates a value close to 0, it means that the two data are not similar. Further, if the correlation coefficient indicates a value close to −1, it means that the signs of the two data are reversed.

$$r = \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}} \\ \overline{x} \text{:} Average value of data x \\ \overline{y} \text{:} Average value of data y \qquad Expression 5$$

Further, the authentication unit 139 may associate the authentication result with an authentication time and the like, and may record the authentication result as an authentication history in the storage unit 141. By generating the above authentication history, it becomes possible to know who requested the vein pattern authentication and when the requester requested the vein pattern authentication, and further who used the vein imaging apparatus 10 and when the user used the vein imaging apparatus 10.

Further, when authentication of a vein pattern obtained from a certain user has failed for a predetermined number of times or more, the authentication unit 139 transmits, to the warning unit 131, a message indicating that the authentication processing has failed for the predetermined number of times or more. By transmitting such information to the warning unit 131, when it is determined that a normal vein authentication processing may not be performed due to thermal expansion caused by an environmental temperature and the like, it is possible to warn the user of the vein imaging apparatus 10 that the authentication might not be normally performed.

The storage unit 141 stores registered vein patterns of the users of the vein imaging apparatus 10 and other data associated with the registered vein patterns. In addition to these data, the storage unit 141 may store the vein image data generated by the imaging unit, the vein image generated by the vein image interpolation unit 133, and vein pattern and the like extracted by the vein pattern extraction unit 137. In addition, in the storage unit 141 may be stored various programs, data, and the like that are needed in the interpolation processing performed by the vein image interpolation unit 135. Further, in addition to these data, the storage unit 141 may store various parameters or progress of processing that are necessary to be stored while the vein imaging apparatus 10 performs certain processing, various kinds of databases or the like. This storage unit 141 can be freely read and written by each processing unit included in the imaging unit, the image processing unit, and the authentication processing unit.

[Regarding Obtaining Data from Particular Pixel]

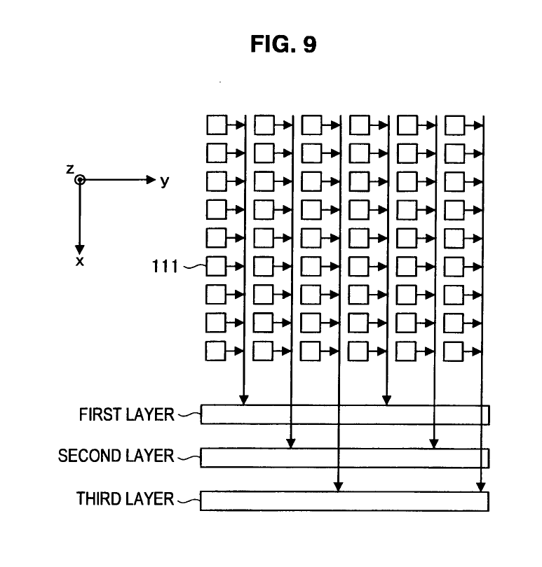

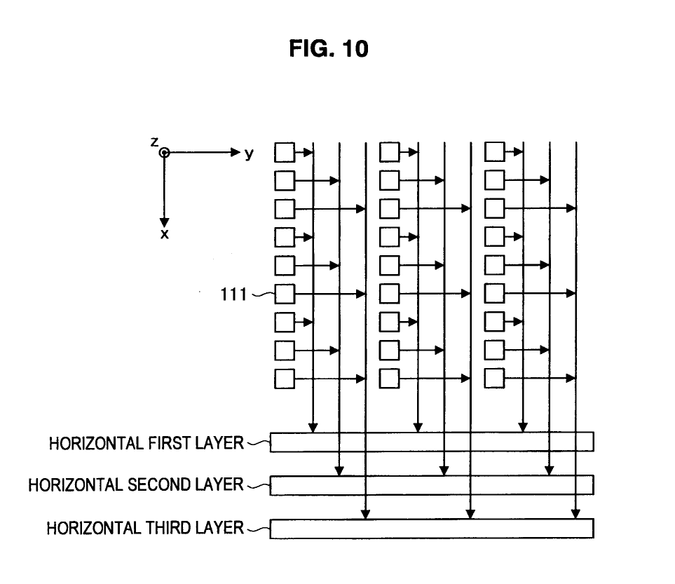

A method of obtaining data from a particular pixel will be hereinafter described in detail with reference to FIG. 9 and FIG. 10. FIG. 9 and FIG. 10 are explanatory diagrams for illustrating the method for obtaining data from the particular pixel.

The imaging element 109 of the vein imaging apparatus 10 according to the present embodiment is a multi-layer element. For example, FIG. 9 shows an example of a case where the imaging element 109 is a multi-layer element made of three layers.

In the vein imaging apparatus 10 according to the present embodiment, the imaging element 10 performs line-scanning in a longitudinal direction of a finger, namely, in a direction along y-axis in the figure. Hereinafter, the direction along the y-axis in the figure will be referred to as vertical direction. A direction perpendicular to the vertical direction, namely, a direction along x-axis in the figure will be referred to as horizontal direction.

As shown in FIG. 9, in the vein imaging apparatus 10 according to the present embodiment, image data is output by the drive control unit 121 in units of horizontal lines along a temporal axis of vertical synchronization. In other words, data for some pixels disposed along the horizontal direction is output to the first layer shown in FIG. 9 in synchronization, data for some pixels disposed along the horizontal direction output to the second layer, and data for some pixels disposed along the horizontal direction is output to the third layer. In this manner, according to the control of the drive control unit 121, the imaging element 109 can output with the multiple layers.

Therefore, it becomes possible for the pixel selection unit 133 to transmit information about the pixels to be selected to the drive control unit 121, and for the drive control unit 121 to select an output with a certain layer of the multi-layer element and to select a particular pixel on the horizontal line by the timing control.

In the example shown in FIG. 9, the method for divisionally driving the vertical synchronization line has been described. Alternatively, as shown in FIG. 10, it may also be possible to perform divisional driving within the horizontal line by means of a circuit.

In the example shown in FIG. 10, there are three types of the pixels 111 on the same horizontal line, i.e., those which output data to the first horizontal layer, those which output data to the second horizontal layer, and those which output data to the third horizontal layer. Therefore, by selecting an output with a certain layer of the multi-layer element and performing timing control for selecting a particular pixel on the vertical line, the drive control unit 121 can select data provided by any pixel.

Alternatively, the divisional driving within the vertical line and the divisional driving within the horizontal line may be used in combination.

An example of the function of the vein imaging apparatus 10 according to the present embodiment has been described in the foregoing. Each of the above-described elements may be constituted using a general-purpose member or circuit, or it may be constituted by the hardware specialized to the function of each element. Further, the function of each element may be entirely realized by a CPU or the like. It is thereby possible to change the configuration to be used as appropriate according to the technique level when implementing the embodiment.

Besides, it is possible to make a computer program for implementing each of the functions of the above-described vein imaging apparatus according to the present embodiment, and to implement the computer program in a personal computer and the like that can control an imaging apparatus having a microlens array, a near-infrared light emission source, and an imaging element. A computer-readable recording medium in which the above computer program is stored may also be provided. The recording medium may be, for example, a magnetic disk, an optical disk, a magneto-optical disk, and a flash memory. Alternatively, the above computer program may be distributed via, e.g., a network instead of using the recording medium.

The vein imaging apparatus 10 according to the present embodiment may be implemented in an information processing apparatus such as a computer or a server, a mobile terminal such as a cellular phone or a PHS or a portable information terminal (PDA), an automated-teller machine (ATM), an access management apparatus. Further, the vein imaging apparatus 10 according to the present embodiment may be implemented in various kinds of apparatuses such as a game machine, a controller of a game machine or the like.

In the above explanation, the registered vein patterns previously registered as templates are assumed to be recorded in the vein imaging apparatus 10. Alternatively, the registered vein patterns may be stored in a recording medium such as a DVD medium, a Blu-ray medium, a compact flash (registered trademark), a memory stick, or an SD memory card, an IC card or an electronic device equipped with a contactless IC chip or the like, or may be stored in a server that is connected to the vein imaging apparatus 10 via a communication network such as the Internet.

### <Vein Image Interpolation Method>

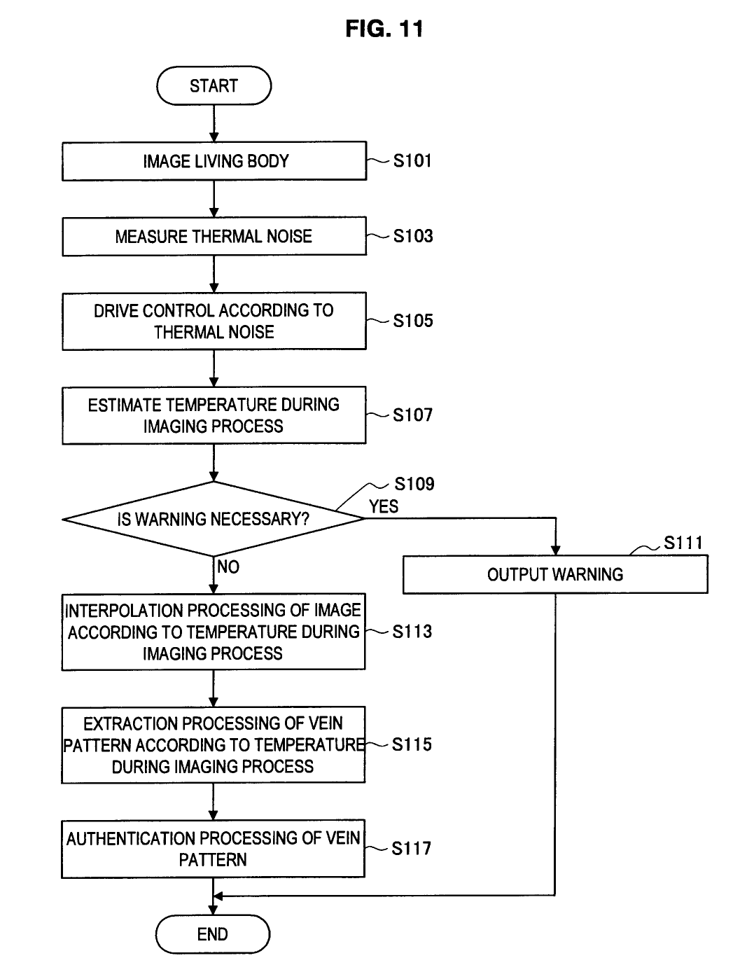

Next, the vein image interpolation method performed by the vein imaging apparatus according to the present embodiment will be described in detail with reference to FIG. 11. FIG. 11 is a flow diagram for illustrating the vein image interpolation method according to the present embodiment.

First, the user of the vein imaging apparatus 10 places a part of the living body such as a finger on the microlens array 101 of the vein imaging apparatus 10. The imaging unit of the vein imaging apparatus 10 performs an imaging processing of the part of the living body placed thereon (step S101).

Further, the thermal noise output preprocessing unit 125 of the vein imaging apparatus 10 performs accumulation processing for accumulatively adding thermal noise output data for a predetermined period of time and a peak processing performed on the thermal noise output data, for the data output from the thermal noise output data generation region 153 of the imaging element 109. Further, the thermal noise measuring unit 127 performs measuring processing of the thermal noise based on the thermal noise output data transmitted from the thermal noise output preprocessing unit 125 (step S103). The thermal noise measuring unit 127 transmits the measurement result of the thermal noise to the drive control unit 121 and the temperature estimation unit 129. Here, the drive control unit 121 performs drive control of the imaging element 109 according to the measuring result of the transmitted thermal noise (step S105).

Subsequently, the temperature estimation unit 129 estimates the temperature during imaging process based on the measuring result of the thermal noise transmitted from the thermal noise measuring unit 127 (step S107). In addition, the temperature estimation unit 129 may also estimate the degree of the decrease of the S/N ratio based on the estimated temperature. The temperature estimation unit 129 transmits the temperature estimation result and the information about the temperature including the estimation result and the like of the degree of the decrease of the S/N ratio to the warning unit 131, the pixel selection unit 133, the vein image interpolation unit 135, and the vein pattern extraction unit 137.

The warning unit 131, to which the information about the temperature was transmitted, makes a determination on the estimation result of the temperature (step S109), and determines whether the temperature exceeds the threshold value at which a warning is required. When the temperature reaches the level at which a warning is required, the vein imaging apparatus 10 outputs a warning on a display screen (step S111).

When the temperature has not yet reached the level at which a warning is required, the pixel selection unit 133 performs selection processing of pixels based on the received information about the temperature. More specifically, the pixel selection unit 133 selects a pixel outputting image data used for generating the vein image from among the plurality of pixels corresponding to one of the microlenses 103, for each of the microlenses 103 constituting the microlens array 101.

Next, the vein image interpolation unit 135 generates the vein image by using the image data obtained from the pixel selected by the pixel selection unit 133. Subsequently, the vein image interpolation unit 135 performs image interpolation processing for the generated vein image according to the temperature during imaging process (step S113). More specifically, the vein image interpolation unit 133 performs integration processing of the plurality of frame images, denoising processing, and interpolation processing of the image using the neighboring pixels.

When the interpolation processing of the image is completed, the vein image interpolation unit 135 transmits the vein image on which the interpolation processing was performed to the vein pattern extraction unit 137. The vein pattern extraction unit 137 extracts the vein pattern from the transmitted vein image (step S115) by changing a filter characteristic of a filter for extracting the vein pattern according to the temperature at the time of imaging, and transmits the extracted vein pattern to the authentication unit 139.

The authentication unit 139 performs authentication processing of the transmitted vein pattern by using the vein pattern transmitted from the vein pattern extraction unit 137 and the registered vein patterns (templates) stored in the storage unit 141 and the like (step S115).

According to the above-described procedure, the deterioration of the image quality caused by the thermal noise at high temperature can be automatically interpolated.

In the above explanation, the measuring processing of the thermal noise and the estimation processing of the temperature during imaging process are carried out after the imaging processing of the living body. Alternatively, the vein imaging apparatus 10 may previously perform the measuring processing of the thermal noise and the estimation processing of the temperature during imaging process before performing the imaging processing of the living body.

### [Regarding Hardware Configuration]

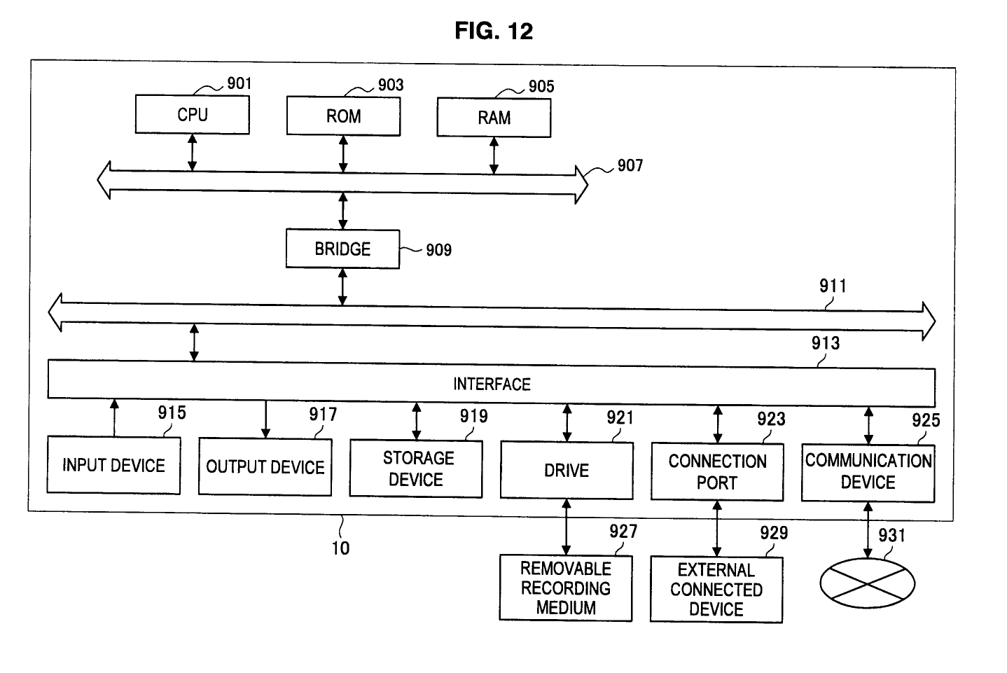

A hardware configuration of the vein imaging apparatus 10 according to an embodiment of the present invention is described hereinafter with reference to FIG. 12. FIG. 12 is a block diagram for illustrating a hardware configuration of the vein imaging apparatus 10 according to an embodiment of the present invention.

The vein imaging apparatus 10 includes the microlens array 101, the near-infrared light emission source 105, and the imaging element 109. In addition, the vein imaging apparatus 10 includes a CPU 901, a ROM 903, and a RAM 905. Further, the vein imaging apparatus 10 includes a host bus 907, a bridge 909, an external bus 911, an interface 913, an input device 915, an output device 917, a storage device 919, a drive 921, a connection port 923, and a communication device 925.

The CPU 901 functions as a processing unit and a control unit, and it controls the whole or a part of operation in the vein imaging apparatus 10 according to various kinds of programs stored in the ROM 903, the RAM 905, the storage device 919 or a removable recording medium 927. The ROM 903 stores a program to be used by the CPU 901, a processing parameter and the like. The ROM 903 stores a program to be used by the CPU 901, a processing parameter and so on. The RAM 905 primarily stores programs used by the CPU 901 in the execution, parameters and the like that are changed during the execution. The CPU 901, the ROM 903 and the RAM 905 are connected with one another through the host bus 907, which is an internal bus such as a CPU bus.

The host bus 907 is connected to the external bus 911 such as a Peripheral Component Interconnect/Interface (PCI) bus via the bridge 909.

The input device 915 is an operating means to be operated by a user, such as a mouse, a keyboard, a touch panel, buttons, a switch or a lever, for example. For example, the input device 915 may be a remote controlling means (or a remote control) with an infrared ray or another radio wave, or an externally connected device 929 compatible with the operation of the vein imaging apparatus 10, such as a cellular phone or a PDA. Further, the input device 915 includes an input control circuit that generates an input signal based on information input by a user using the above operating means and outputs it to the CPU 901, for example. By operating this input device 915, a user of the vein imaging apparatus 10 can input various kinds of data or give an instruction of a processing operation to the vein imaging apparatus 10.

The output device 917 includes an apparatus capable of visually or audibly notifying obtained information to the user. Examples of such apparatus include a display device such as a CRT display device, a liquid crystal display device, a plasma display device, an EL display device or a lamp, an audio output device such as a speaker or a headphone, or a printer, a cellular phone or a facsimile. The output device 917 outputs, for example, results obtained by various processing by the vein imaging apparatus 10. Specifically, the display device displays, as a text or an image, a result obtained by various processing of the vein imaging apparatus 10. The audio output device converts an audio signal containing reproduced audio data, acoustic data or the like into an analog signal and outputs it.

The storage device 919 is a device for data storage that is configured as an example of a storage unit of the vein imaging apparatus 10. The storage device 919 may include a magnetic storage device such as a hard disk drive (HDD), a semiconductor storage device, an optical storage device, a magneto-optical storage device or the like. This storage device 919 stores a program to be executed by the CPU 901, various data, or various data acquired from the outside, for example.

The drive 921 is a reader/writer for a recording medium, which is built in the vein imaging apparatus 10 or attached thereto. The drive 921 reads information that is recorded in the removable recording medium 927 such as a magnetic disk, an optical disk, a magneto-optical disk or semiconductor memory which is attached thereto and outputs the information to the RAM 905. Further, the drive 921 can write information into the removable recording medium 927 such as a magnetic disk, an optical disk, a magneto-optical disk or semiconductor memory which is attached thereto. Examples of the removable recording medium 927 include a DVD medium, an HD-DVD medium, and a Blu-ray medium. In addition, examples of the removable recording medium 927 include a compact flash (registered trademark) (CF), a memory stick, and a secure digital (SD) memory card. Further, the removable recording medium 927 may be an integrated circuit (IC) card equipped with a contactless IC chip or an electronic appliance.

The connection port 923 is a port for directly connecting devices to the vein imaging apparatus 10. Examples of the connection port 923 include a universal serial bus (USB) port, an IEEE 1394 port such as i.Link, and a small computer system interface (SCSI) port. In addition, examples of the connection port 923 include an RS-232C port, an optical audio terminal, and a high-definition multimedia interface (HDMI) port. By connecting the externally connected device 929 to the connection port 923, the vein imaging apparatus 10 can directly acquire various data from the externally connected device 929 or supply various data to the externally connected device 929.

The communication device 925 is a communication interface that is constituted by a communication device or the like for connecting to a communication network 931, for example. The communication device 925 may be a communication card for wired or wireless local area network (LAN), Bluetooth, or wireless USB (WUSB). Alternatively, the communication device 925 may be a router for optical communication, a router for asymmetric digital subscriber line (ADSL), or a modem for each kind of communication. This communication device 925 can transmit and receive a signal or the like in conformity to a prescribed protocol such as TCP/IP on the Internet or with other communication devices, for example. Further, the communication network 931 that is connected to the communication device 925 includes a wired or wireless network or the like, and it may be the Internet, home LAN, infrared data communication (network), radio wave communication, satellite communication or the like.

An example of the hardware configuration that can implement the functions of the vein imaging apparatus 10 according to each embodiment of the present invention has been described in the foregoing. Each of the above-described elements may be constituted using a general-purpose member or circuit, or it may be constituted by hardware specialized to the function of each element. It is thereby possible to change the configuration to be used as appropriate according to the technique level when implementing the embodiment.

### SUMMARY

As hereinabove described, according to each embodiment of the present invention, it is possible to realize the vein imaging apparatus that automatically obtains the operational state of the imaging element so that authentication and imaging of the living body can be performed even when the imaging element is under a severe environment, e.g., at the temperature of 70 degrees Celsius. By automatically obtaining the operational state of the imaging element, it becomes possible to perform adaptive driving method and image signal processing, and image acquisition and authentication processing for biometrics authentication can be achieved independent on the temperature.

The imaging element according to each embodiment of the present invention has in addition to an imaging region for generating vein image data but a region that is shielded from the external light. By detecting noise (thermal noise) in this region, the increase of the noise caused by the operational temperature is detected. Since the vein imaging apparatus according to the present embodiment can estimate relative temperature variation of the apparatus with the detected noise, the driving control method of the imaging element and the image signal processing method can be changed according to this temperature variation information.

The vein imaging apparatus according to each embodiment of the present invention can accurately execute the accumulative addition processing of frames and shutter speed, the image signal processing, and the like by measuring the noise level of the dedicated pixels equivalent to the image sensor. Since the region for detecting noise can be arranged in the imaging element, the structure that does not affect the production cost of the apparatus can be achieved. By time-divisionally importing the data about noise and the taken image data, the driving circuit for importing data about noise can be also used for importing taken image data.

It should be understood by those skilled in the art that various modifications, combinations, sub-combinations and alterations may occur depending on design requirements and other factors insofar as they are within the scope of the appended claims or the equivalents thereof.

The present application contains subject matter related to that disclosed in Japanese Priority Patent Application JP 2009-117986 filed in the Japan Patent Office on May 14, 2009, the entire content of which is hereby incorporated by reference.

What is claimed is:

1. A vein imaging apparatus comprising:
a lens array including a plurality of light-receiving lenses disposed in an array;
a near-infrared light emission source that is arranged at an end of the lens array and emits a near-infrared light to a part of a living body;
an imaging element including:
a vein image data generation region for generating image data of a vein based on the near-infrared light that was condensed by the lens array and that was scattered in the living body and transmitted through the vein; and
a thermal noise output data generation region that includes a pixel shielded from light and generates a thermal noise output that is an output value output from the pixel shielded from light;
a thermal noise measuring unit that measures a magnitude of thermal noise based on the thermal noise output data output from the thermal noise output data generation region;
a temperature estimation unit that estimates an imaging temperature at which imaging processing of the vein is performed based on the magnitude of the thermal noise measured by the thermal noise measuring unit; and
a vein image interpolation unit that generates an image of the vein using the vein image data generated by the vein image data generation region, and performs interpolation processing of the image of the vein based on the imaging temperature estimated by the temperature estimation unit.

2. The vein imaging apparatus according to claim 2, wherein the vein image interpolation unit performs at least one of integration processing of the image of the vein for a predetermined period and denoising processing of the image of the vein based on the imaging temperature estimated by the temperature estimation unit.

3. The vein imaging apparatus according to claim 1,
wherein, in the image element, a plurality of pixels located in the vein image data generation region correspond to one of the light-receiving lenses and
wherein the vein image interpolation unit performs the interpolation processing using the vein image data output from pixels located around a pixel that output the vein image data used for generating the image of the vein.

4. The vein imaging apparatus according to claim 2,
wherein, in the image element, a plurality of pixels located in the vein image data generation region correspond to one of the light-receiving lenses and
wherein the vein image interpolation unit performs the interpolation processing using the vein image data output from pixels located around a pixel that output the vein image data used for generating the image of the vein.

5. The vein imaging apparatus according to claim 1, further comprising a thermal noise output preprocessing unit that performs preprocessing, for allowing the thermal noise measuring unit to quantitatively process the thermal noise, on the thermal noise output data output from the thermal noise output data generation region.

6. The vein imaging apparatus according to claim 4, wherein the thermal noise output preprocessing unit performs at least one of accumulative processing for accumulatively adding the thermal noise output data for a predetermined period and peak processing on the thermal noise output data.

7. The vein imaging apparatus according to claim 1, further comprising a drive control unit that performs drive control of at least the image element,
wherein the drive control unit controls at least one of a light-receiving time and a frame rate of the image element based on the magnitude of the thermal noise measured by the thermal noise measuring unit.

8. The vein imaging apparatus according to claim 1, further comprising a vein pattern extraction unit that extracts a vein pattern from the image of the vein,
wherein the vein pattern extraction unit changes a filter characteristic of a filter used for extracting the vein pattern based on the imaging temperature estimated by the temperature estimation unit.

9. The vein imaging apparatus according to claim 1, further comprising a warning unit for giving a warning when the imaging temperature output by the temperature estimation unit is equal to or more than a predetermined threshold value.

10. A vein image interpolation method comprising the steps of:
measuring a magnitude of thermal noise based on a thermal noise output data output from a thermal noise output data generation region of a vein imaging apparatus including a lens array including a plurality of light-receiving lenses disposed in an array, a near-infrared light emission source that is arranged at an end of the lens array and emits a near-infrared light to a part of a living body, and an image element including a vein image data generation region for generating image data of a vein based on the near-infrared light that was condensed by the lens array and that was scattered in the living body and transmitted through the vein and the thermal noise output data generation region that includes a pixel shielded from light and generates a thermal noise output that is an output value output from the pixel shielded from light;
estimating an imaging temperature at which an imaging processing of the vein is performed based on the magnitude of the measured thermal noise; and
generating an image of the vein using the vein image data generated by the vein image data generation region, and performing interpolation processing of the image of the vein based on the estimated imaging temperature.

11. A program for causing a computer that controls a vein imaging apparatus to realize:
a thermal noise measuring function for measuring a magnitude of thermal noise based on a thermal noise output data output from a thermal noise output data generation region;
a temperature estimation function for estimating an imaging temperature at which an imaging processing of a vein is performed based on the magnitude of the thermal noise measured by the thermal noise measuring function; and
a vein image interpolation function for generating an image of the vein using vein image data generated by a vein image data generation region, and performing interpolation processing of the image of the vein based on the imaging temperature estimated by the temperature estimation function,
wherein the vein apparatus includes a lens array including a plurality of light-receiving lenses disposed in an array, a near-infrared light emission source that is arranged at an end of the lens array and emits a near-infrared light to a part of a living body, and an image element including the vein image data generation region for generating image data of a vein based on the near-infrared light that was condensed by the lens array and that was scattered in the living body and transmitted through the vein and a thermal noise output data generation region that includes a pixel shielded from light and generates a thermal noise output that is an output value output from the pixel shielded from light.
